(19) United States
(12) Patent Application Publication
(10) Pub. No.: US 2019/0388069 A1
(43) Pub. Date: Dec. 26, 2019

(54) NEEDLE ASSEMBLY AND SAMPLE ANALYSIS SYSTEM FOR COLLECTION AND OPTICAL INTERROGATION OF A BIOLOGICAL SAMPLE

(71) Applicant: INSTITUT NATIONAL D'OPTIQUE, Quebec (CA)

(72) Inventors: Jessie WEBER, Quebec (CA); Christophe RIVIERE, Quebec (CA); Antoine PROULX, Quebec (CA)

(73) Assignee: INSTITUT NATIONAL D'OPTIQUE, Quebec, QC (CA)

(21) Appl. No.: 16/481,317

(22) PCT Filed: Jan. 26, 2018
(86) PCT No.: PCT/CA2018/050094

§ 371 Date: Jul. 26, 2019

Related U.S. Application Data
(60) Provisional application No. 62/451,302, filed on Jan. 27, 2017.

(51) Int. Cl. A61B 10/02; A61B 5/00

(52) U.S. Cl. CPC A61B 10/0233; A61M 5/32; A61B 5/6848; A61B 5/0075

(57) ABSTRACT

Needle assemblies and analysis systems for collection and optical interrogation of a biological sample is provide. The needle assembly includes a needle hub and a needle tip. A shaft portion extends between the needle hub and the needle tip. The shaft portion includes a cavity extending from the needle hub to the needle tip. The cavity includes a sample-receiving region opened at the needle tip. In some embodiments, the shaft portion includes a cladding structure surrounding the cavity and configured for longitudinal light guidance. In other embodiments, the shaft portion includes an optical window in line-of-sight alignment with the sample-receiving region. The needle assembly may advantageously be used to collect, within the sample receiving region, a biological sample from a biological medium and perform an optical interrogation of this sample directly in the needle assembly.

### TECHNICAL FIELD

The technical field generally relates to needles for biopsies and the like, and more particularly concerns a needle assembly providing for the collection of a biological sample and its analysis using optical interrogation techniques as well as sample analysis systems and methods using such a needle assembly.

### BACKGROUND

As is known in the art, optical techniques can be used in the analysis of biological samples, such as for biopsies and the like, in a multitude of fashions.

By way of example, U.S. Pat. No. 9,186,064 (SHUMATE et al) entitled “Internal optical spectroscope and method for real time in-situ diagnosis in living cells” teaches an approach to make optical measurements in living tissue. Similarly, U.S. Pat. No. 9,179,845 (FARCY et al) entitled “Sharp fibrous needle probe for the in-depth optical diagnostics of tumours by endogenous fluorescence” discloses in vivo optical diagnosis and screening. No sample is collected in both cases, the tissues being optically interrogated in situ of the subject. Optical techniques are also known to guide biopsies, as disclosed for example in International Pat. Appl. Pub. No. WO 2014/068468 to BIERHOFF et al (“System with photonic biopsy device for obtaining pathological information”).

It is also known in the art to use a needle to collect a tissue to be biopsied. Techniques for needle biopsies can be divided into two main types: Fine Needle Aspiration (FNA), where a small needle (21-25 gauge is typical) is used to collect tissue, typically in palpable tumors; and Core Needle Biopsy (CNB) where a larger, and more invasive needle is used to collect and extract a larger sample for analysis. All-optical needle biopsy techniques, in which an optical measurement replaces the physical biopsy, are also known in the art. Advantageously, such techniques provide information without the need to remove a sample from the subject. However, the absence of a collected sample precludes performing further standard analysis after the initial measurement.

It is also known in the art to perform optically-guided needle biopsies and optically-guided surgical tumor resections. In such approaches an optical measurement is used to guide the physical biopsy, as a form of pre-screening to confirm areas of interest. Optical guiding can avoid damaging normal tissues as it precludes the need to perform a physical biopsy to test for abnormality. Measurements made in bulk tissues can however be subject to background noise, and validation that the optical measurement was taken in exactly the location and volume of the biopsy can be difficult.

Ex vivo optical biopsy or measurements remain a widespread practice. Tissues or other biological samples are collected from the body of the subject using a standard needle, transferred to a suitable support medium and an optical measurement is taken. Typically, the sample is then sent for further analysis, such as pathology. Performing such optical analysis immediately or shortly after the collection of the sample can provide useful feedback on positive vs. negative margins during surgical procedures, i.e. let the surgeon know if an entire tumor was removed (negative margins) or if cancer cells remain in the body (positive margins). Unfortunately, such an approach has some drawbacks. Firstly, ex vivo margin assessment techniques involve extra handling of the collected tissues, which can dry out or otherwise be compromised. Another drawback is defining and maintaining fiducials to validate location with pathology.

There remains a need for devices and methods that improve on at least some of the above-mentioned techniques.

### SUMMARY

In accordance with one aspect, there is provided a needle assembly for collection and optical interrogation of a biological sample.

In some implementations, the needle assembly includes a needle hub and a needle tip. A shaft portion extends between the needle hub and the needle tip. The shaft portion includes a cavity extending from the needle hub to the needle tip. The cavity includes a sample receiving region opened at the needle tip.

In some embodiments, the shaft portion includes a cladding structure surrounding the cavity. In some configurations the needle assembly may be configured for light guidance along an optical axis extending along the longitudinal axis of the shaft portion to perform an optical interrogation of the sample in the sample-receiving region.

In other embodiments, the shaft portion includes a capillary having a longitudinal cavity. The shaft portion further includes an optical window transversally aligned with the sample-receiving region so as to allow optical interrogation of the sample within the sample receiving region transversally to the longitudinal axis of the shaft portion.

In some implementations, the needle assembly may advantageously be used to collect, within the sample-receiving region, a biological sample from a biological medium and perform an optical interrogation of this sample directly in the needle assembly. In some implementations, the optical interrogation may be performed in situ of the patient immediately or shortly after the sample is drawn into the needle assembly. In other implementations, optical interrogation of the sample within the needle assembly may be performed subsequently to its removal from the sampling site.

In accordance with some implementations, there is also provided a sample analysis system making use of needle assemblies as described herein or equivalents thereof.

In accordance with further implementations, there are also provided methods for the diagnosis, prognosis or treatment of a disease or a condition in a subject involving the use of a needle assembly or analysis system such as described herein.

According to one aspect, there is provided a needle assembly for collection and optical interrogation of a biological sample. The needle assembly includes:
a needle hub;
a needle tip; and
a shaft portion disposed longitudinally between the needle hub and the needle tip, the shaft portion comprising a cavity extending from the needle hub to the needle tip, the cavity comprising a sample-receiving region opened at the needle tip for collection of the biological sample through said needle tip, the shaft portion further comprising a cladding structure surrounding said cavity, the shaft portion being configured for light guidance therein along a longitudinal optical axis to perform an optical interrogation of the sample in the sample-receiving region.

In accordance with some implementations, the cavity and the cladding structure have jointly beveled extremities at the needle tip. The needle assembly may further include a sheath surrounding the shaft portion and having a beveled extremity at the needle tip. In accordance with some implementations, the sheath is made of a biocompatible material.

In accordance with some implementations, the shaft portion further includes one or more coating layers surrounding the cladding structure. Each of the one or more coating layers may be made of a polyimide, an acrylate, a low-index polymer, silicon or a metal.

In accordance with some implementations, the cladding structure includes an optical cladding layer made of an optical material, for example silica, suitable for light propagation therein. The optical cladding layer of the cladding structure is preferably contiguous to the cavity, and the cladding structure may further include an air hole layer extending within said optical cladding layer in an air-clad configuration, the optical cladding layer defining an interstitial ring between the cavity and the air hole layer, the interstitial ring providing said light guidance.

In accordance with some implementations, the cladding structure further includes an optical fiber core extending within the optical cladding layer and parallel to said cavity, the optical fiber core providing said light guidance. The optical fiber core may have an elliptical cross section. In some variants, the cavity is positioned eccentrically with respect to a central axis of the shaft portion and the optical fiber core extends along said central axis.

In accordance with some implementations, the cladding structure further includes a plurality of optical fiber cores extending within the optical cladding layer in parallel to the cavity and distributed around said cavity, the plurality of optical fiber cores defining an array of light paths providing said light guidance. Alternatively, the cladding structure may include an integrated optical fiber having an optical fiber core and an optical fiber cladding, the integrated optical fiber having a longitudinal surface polished through to the optical fiber core and extending in longitudinal contact with said cavity. In yet another set of variants, the cladding structure may include one or more partial optical fiber cores extending in longitudinal contact with the cavity.

In accordance with some implementations, the cladding structure includes, concentrically and outwardly from the cavity:
a ring core layer;
a low refractive index cladding layer; and
a high refractive index cladding layer;
   whereby said ring core layer provides said light guidance.

In accordance with some implementations, the needle assembly further includes a reflective coating deposited on an extremity of the cladding structure at the needle tip.

In accordance with another aspect, there is provided a sample analysis system for collection and optical interrogation of a biological sample.

The sample analysis system includes a needle assembly according to one of the variants described above. The sample analysis system further includes an optical assembly including a light source generating an interrogation light beam, the light source being connectable to the needle hub so as to allow optical coupling of the interrogation light beam into the cladding structure of the shaft portion of the needle assembly for light guidance therein along the longitudinal optical axis of the shaft portion, the optical assembly further comprising an optical detector connectable to the needle assembly to detect light resulting from an interaction of said interrogation light beam with the biological sample.

In accordance with some implementations, the light source of the optical assembly includes a plurality of light source components collectively generating the interrogation light beam. The light source of the optical assembly may for example include at least one of a broadband light source, a LED or a laser.

In accordance with some implementations, the optical detector includes a spectrometer, a photomultiplier tube or an image capture device.

In accordance with some implementations, the optical assembly further includes at least one input optical fiber link optically coupling the interrogation light beam from the light source to the needle assembly.

In accordance with some implementations, the optical assembly further includes at least one output optical fiber link optically coupling the light resulting from an interaction of said interrogation light beam with the biological sample from the needle assembly to the optical detector.

In accordance with some implementations, the optical assembly includes:
at least one input optical fiber link optically coupling the interrogation light beam from the light source to the needle assembly;
at least one output optical fiber link optically coupling the light resulting from an interaction of said interrogation light beam with the biological sample from the needle assembly to the detector; and
an optical coupler comprising a first connector affixed to the needle hub and a second connector engageable with the first connector and housing extremities of said input and output optical fiber links.

In accordance with some implementations, the optical assembly further includes an optical reader comprising a cap shaped to fit over the needle tip of the needle assembly, the detector being affixed within said cap and positioned to receive light exiting from the needle assembly at the needle tip when said needle tip is inserted in said cap.

In accordance with some implementations, the sample analysis system further includes a syringe assembly connectable to the needle hub of the needle assembly so as to provide a suction force to draw the biological sample into the sample-receiving region of the shaft portion of the needle assembly.

In accordance with another aspect, there is provided a needle assembly for collection and optical interrogation of a biological sample. The needle assembly includes:
a needle hub;
a needle tip; and
a shaft portion disposed longitudinally between the needle hub and the needle tip, the shaft portion comprising a capillary having a cavity extending longitudinally from the needle hub to the needle tip, the cavity comprising a sample-receiving region opened at the needle tip for collection of the biological sample through said needle tip, the capillary further comprising at least one optical window in line of sight alignment with the sample-receiving region and allowing optical interrogation of the sample within the sample-receiving region therethrough.

In accordance with some implementations, the capillary is made of a transparent material, a portion thereof defining each of the at least one optical window. The transparent material may for example be silica or a plastic.

In accordance with some implementations, the the at least one optical window includes an input window and an output window. The input and output windows are preferably provided on opposite sides of the capillary in optical alignment.

In accordance with some implementations, the needle assembly further includes a sheath made of a biologically compatible material surrounding the shaft portion. The biocompatible material may for example include a metal or a polyimide. In some variants, the sheath has a bevelled edge defining the needle tip. Furthermore, the sheath is may be movable over the capillary between a sampling position enabling drawing of the sample inside the sample-receiving region and a retracted position exposing the at least one optical window to optical interrogation therethrough. In other variants, the sheath may be made of a transparent material.

The sheath may alternatively include at least an opening or transparent inclusion optically aligned with the at least one optical window of the capillary.

In accordance with yet another aspect, there is provided a sample analysis system for collection and optical interrogation of a biological sample, including a needle assembly according to the aspect just described above.

The sample analysis system includes an optical reader which itself includes:
a reading chamber sized to receive the needle tip therein;
at least one light source generating one or more interrogation light beams and configured to project said interrogation light beams on the biological sample in the sample-receiving region through the at least one optical window when the needle tip is inserted into the reading chamber; and
at least one optical detector positioned and configured to detect light resulting from an interaction of the biological sample with said one or more interrogation light beams.

In accordance with some implementations, the at least one light source and the at least one optical detector are disposed on opposite sides of the reading chamber. In other implementations, the at least one light source and the at least one optical detector are disposed on a same side of the reading chamber.

In accordance with some implementations, the sample analysis system further includes a syringe assembly connectable to the needle hub so as to provide a suction force to draw the biological sample into the sample-receiving region of shaft portion of the needle assembly.

In accordance with another aspect, there is provided a needle assembly for collection and optical interrogation of a biological sample, including:
a needle hub;
a needle tip; and
   a shaft portion disposed longitudinally between the needle hub and the needle tip, the shaft portion comprising a cavity extending longitudinally from the needle hub to the needle tip, the cavity comprising a sample-receiving region opened at the needle tip for collection of the biological sample through said needle tip, the shaft portion being configured to allow optical interrogation of the sample within the sample-receiving region.

In accordance with some implementations, the shaft portion has at least one of a longitudinal optical interrogation configuration and a transversal optical interrogation configuration.

The longitudinal optical interrogation configuration may include a cladding structure surrounding the cavity and providing light guidance therein along a longitudinal optical axis of the shaft portion. the cladding structure for example includes an optical cladding layer made of an optical material and configured to provide said light guidance therein. The shaft portion may further include at least one optical fiber core extending within the optical cladding layer parallel to said cavity, the optical fiber core being configured to provide said light guidance therein.

The transversal optical interrogation configuration may include at least one optical window provided in the shaft portion in line of sight alignment with the sample-receiving region and allowing said optical interrogation of the sample within the sample-receiving region therethrough. The needle assembly may further include a sheath made of a biocompatible material surrounding the shaft portion, the sheath being movable over the shaft portion between a sampling position enabling drawing of the sample inside the sample-receiving region and a retracted position exposing the at least one optical window to optical interrogation therethrough.

In accordance with yet another aspect, there is provided a sample analysis system for collection and optical interrogation of a biological sample, including:
a needle assembly comprising a needle hub, a needle tip and a shaft portion disposed longitudinally between the needle hub and the needle tip, the shaft portion comprising a cavity extending longitudinally from the needle hub to the needle tip, the cavity comprising a sample-receiving region opened at the needle tip for collection of the biological sample through said needle tip, the shaft portion being configured to allow optical interrogation of the sample within the sample-receiving region; and
an optical assembly for optical interrogation of the sample within the sample-receiving region, the optical assembly comprising:
   at least one light source generating an interrogation light beam and configured to optically interrogate the biological sample in the sample-receiving region with said interrogation light beam; and
   at least one optical detector positioned and configured to detect light resulting from an interaction of the biological sample with said interrogation light beam.

In accordance with some implementations, the sample analysis system further includes a syringe assembly connectable to the needle hub of the needle assembly so as to provide a suction force to draw the biological sample into the sample-receiving region of the shaft portion of the needle assembly.

In accordance with some implementations, each of the at least one light source comprises a broadband light source, a LED or a laser.

In accordance with some implementations, each of the at least one optical detector comprises a spectrometer, a photomultiplier tube or an image capture device.

In accordance with some implementations, the shaft portion of the needle assembly lay include a cladding structure surrounding the cavity and providing light guidance therein along a longitudinal optical axis of the shaft portion, and the optical assembly may include at least one input optical fiber link optically coupling the interrogation light beam from the light source to the cladding structure of the needle assembly.

In accordance with some implementations, the optical assembly further includes an optical reader comprising a cap shaped to fit over the needle tip of the needle assembly, the detector being affixed within said cap and positioned to receive light exiting from the needle assembly at the needle tip when said needle tip is inserted in said cap.

In accordance with some implementations, the needle assembly includes at least one optical window provided in the shaft portion in line of sight alignment with the sample-receiving region and allowing said optical interrogation of the sample within the sample-receiving region therethrough, and the sample analysis system includes an optical reader having a reading chamber sized to receive the needle tip therein and incorporating said optical assembly.

In accordance with another aspect, there is provided a use of the needle assembly according to some of the embodiments described above, for an optical interrogation of a biological sample, wherein the sample is in the sample-receiving region of said needle assembly during said optical interrogation. In accordance with some implementations, the biological sample is from a subject's body, and said optical analysis is carried out in situ or ex vivo of the subject's body. The biological sample may for example be a tissue or a biological fluid.

In accordance with another aspect, there is provided a use of the needle assembly according to some of the embodiments described above, for an optical analysis of a biological sample of a subject to aid in diagnosis, or guide treatment of, a disease or condition in said subject, wherein said sample is in said sample-receiving region of said needle assembly during said optical analysis. The optical analysis may be carried out in situ or ex vivo of the subject. The biological sample is for example a tissue or a biological fluid. The sample is preferably in the sample-receiving region of said needle assembly during said optical interrogation. In some embodiments, the biological sample is from a subject's body, and said optical analysis is carried out ex vivo of the subject's body. The biological sample is for example a tissue or a biological fluid.

In accordance with yet another aspect, there is provided a method for analyzing a biological sample, comprising the steps of:
obtaining a needle assembly according to one of the embodiments described above;
inserting the needle tip of the needle assembly in a target site comprising a biological sample;
collecting the biological sample from said target site in the sample-receiving region of the shaft portion of the needle assembly;
optically interrogating the biological sample within the sample-receiving region of the shaft portion of the needle assembly using an interrogation light beam; and
detecting and analyzing light resulting from an interaction of the biological sample with said interrogation light beam.

According to another aspect, there is provided a method to aid in the diagnosis, prognosis or to guide treatment of a disease or a condition in a subject, comprising the steps of:
obtaining a needle assembly as defined in one of the embodiments described above;
inserting the needle tip of said needle assembly in a body of the subject such that said needle tip reaches a target site; and
collecting a biological sample from said target site in the sample-receiving region of the shaft portion of the needle assembly;
optically interrogating the biological sample within the sample-receiving region of the shaft portion of the needle assembly using an interrogation light beam;
detecting light resulting from an interaction of the biological sample with said interrogation light beam;
analyzing said light to determine therefrom at least one characteristic specific of said disease or condition; and
transmitting said at least one characteristic specific of said disease or condition to an instrument or a physician.

In some implementations, the step of optically interrogating the biological sample in the methods above may involve optically coupling the interrogation light beam into the cladding structure of the shaft portion of the needle assembly at the needle hub for light guidance in said cladding structure along the longitudinal optical axis of the shaft portion.

In some implementations, the step of collecting a biological sample in the methods above may involve drawing said biological sample within the sample-receiving region using a syringe assembly connected to the needle hub of the needle assembly.

In some implementation, the methods above may involve a step of withdrawing the needle tip from said body part of the patient between the steps of collecting the biological sample and optically interrogating said biological sample.

In accordance with some implementations of in the methods above, the biological sample is a tissue or a biological fluid.

In some implementations, the analyzing step in the methods above may involve using an optical analysis technique selected from visible or near-infrared brightfield or fluorescence microscopy, visible or near-infrared optical coherence tomography, Raman spectroscopy, autofluorescence measurements, diffuse reflectance spectroscopy and refractive index measurements.

Needle assemblies and sample analysis systems according to embodiments of the present description may be of use in a variety of contexts. In some implementations, the needle assembly may be used to perform a biopsy-type analysis, where biological samples such as tissues, blood, cells or biological liquids need to be collected for further analysis, such as pathology, cytology, histology, etc. The expression “optical interrogation” can be understood to refer to one of any number of techniques involving the interaction of an interrogation light beam with the sample and extracting information from light reflected, transmitted, absorbed, emitted or otherwise resulting from this interaction. Embodiments of the needle assembly described herein may be used in conjunction with a variety of optical analysis techniques such as visible or near-infrared brightfield or fluorescence microscopy, visible or near-infrared optical coherence tomography, Raman spectroscopy, autofluorescence measurements, diffuse reflectance spectroscopy, refractive index measurements, evanescent wave sensing, and the like. Advantageously, embodiments of the needle assembly described herein allow optical measurements to be made on the sample directly in the needle assembly, providing for rapid testing and ensuring that the sample has not been damaged or otherwise transformed through its transfer to a different support medium.

At least some implementations of the devices and methods described herein may improve on one or more of the following aspects of known techniques for the collection and analysis of biological samples: providing additional information from an optical measurement with minimal impact to the work flow of the biopsy procedure; minimizing or eliminating extra handling of the tissue to perform the optical measurement; ensuring that the exact collected tissue is interrogated; inherent validation of the tissue sampled between the optical measurement with pathology; and providing a geometry that minimizes unwanted background signal. Furthermore, some implementations may allow for pre-screening of sample adequacy for further analysis such as pathology, and ensures that the exact collected tissue is interrogated. Another opportunity using some embodiments is to improve standard FNA adequacy rates by optically analysing the contents of the needle assembly, allowing for more sample to be collected immediately, as needed to ensure sufficient sample is collected.

Embodiments of the needle assembly described herein may be disposable and suitable for manufacturing using existing systems.

Other features and advantages of the invention will be better understood upon a reading of preferred embodiments thereof with reference to the appended drawings.

### BRIEF DESCRIPTION OF THE DRAWINGS

FIG. 1 is a side elevation view of a needle assembly for longitudinal optical interrogation according to an embodiment; FIG. 1A is a cross-sectional view along line 1A-1A of FIG. 1; FIG. 1B is a cross-sectional view along line 1B-1B of FIG. 1.

FIGS. 2A and 2B are schematized representations of steps of a sampling process using a needle assembly such as shown in FIG. 1.

FIG. 3 is a side elevation view of a needle assembly for longitudinal optical interrogation according to another embodiment; is a cross-sectional view along line 3A-3A of FIG. 3; FIG. 3B is a cross-sectional view along line 3B-3B of FIG. 3.

FIGS. 4A and 4B are respectively a schematized transversal cross-sectional view and an image of a needle assembly including an air-clad configuration.

FIG. 5A is a schematized transversal cross-sectional view of a needle assembly including an integrated elliptical optical fiber core; FIG. 5B is an image of a needle assembly embodying the configuration schematized in FIG. 5A; FIG. 5C is an enlarged view of the elliptical optical fiber core of the needle assembly of FIG. 5B; FIGS. 5D to 5G are schematized transversal cross-sectional views of needle assembly configurations incorporating one or more optical fiber cores.

FIG. 6 is a schematized transversal cross-sectional view of a needle assembly having a multi-layered cladding structure.

FIG. 7 is a side view of an analysis system including a needle assembly for longitudinal optical interrogation according to one variant; FIG. 7A is a side elevation representation of connectors of an optical coupler for use in the analysis system of FIG. 7. FIG. 7B is a front view of one of the connectors of FIG. 7A.

FIG. 8 is a side view of an analysis system including a needle assembly for longitudinal optical interrogation according to another variant.

FIG. 9 is a side elevation view of a needle assembly for transversal optical interrogation according to an embodiment.

FIG. 10 is a side view of an analysis system including a needle assembly for transversal optical interrogation according to one variant.

FIG. 11A is a side elevation view of a needle assembly for transversal optical interrogation according to an embodiment, including a retractable sheath; FIG. 11B is a side view of an analysis system including an optical reader for interrogating the needle assembly of FIG. 11A.

### DETAILED DESCRIPTION

In accordance with some implementations, there is provided a needle assembly for collection and optical interrogation of a biological sample. As will be readily understood from the description below, the use of a needle assembly as described herein may advantageously allow a health practitioner to draw a biological sample from a patient, and perform an optical interrogation of the sample directly in the needle assembly. In some implementations, the optical interrogation may be performed immediately or shortly after the sample is drawn into the needle assembly. In some implementations, the optical interrogation may be performed in situ of the target sampling site, such as for example, a subject's or patient's body, while the needle assembly is still in the target site or subject's body. Alternatively, the optical interrogation may be performed immediately or shortly after the needle assembly is withdrawn from the sampling site (ex vivo). Of course, in other implementations, the optical interrogation may be performed in situ of the sampling site, when, for example, the biological sample is a cell culture. In other implementations, optical interrogation of the sample within the needle assembly may be performed subsequently to its removal from the sampling site (ex vivo or in vitro). Methods of using such a needle assembly according to various embodiments will be described in detail further below.

In accordance with some implementations, there is also provided a sample analysis system making use of needle assemblies as described herein or equivalents thereof.

In accordance with further implementations, there are also provided methods for aiding the diagnosis, prognosis or for guiding treatment of a disease or a condition in a subject involving the use of a needle assembly or analysis system such as described herein.

Needle assemblies and sample analysis systems according to embodiments of the present description may be of use in a variety of contexts. In some implementations, the needle assembly may be used to perform a biopsy-type analysis, where biological samples such as tissues, blood, cells or biological liquids need to be collected for further analysis, such as pathology, cytology, histology, etc. The expression “optical interrogation” can be understood to refer to one of any number of techniques involving the interaction of an interrogation light beam with the sample and extracting information from light reflected, transmitted, scattered, absorbed, emitted or otherwise resulting from this interaction. Embodiments of the needle assembly described herein may be used in conjunction with a variety of optical analysis techniques such as visible or near-infrared brightfield or fluorescence microscopy, visible or near-infrared optical coherence tomography, Raman spectroscopy, autofluorescence measurements, diffuse reflectance spectroscopy, refractive index measurements, evanescent wave sensing, and the like. Advantageously, embodiments of the needle assembly described herein allow optical measurements to be made on the sample directly in the needle assembly, providing for rapid testing and ensuring that the sample has not been damaged or otherwise transformed through its transfer to another support medium. This approach allows for the sample being analyzed to be the same sample as collected while still enabling the subsequent transfer of the sample to a support such as a microscope slide for sample analysis with more conventional means.

### Examples of Needle Assemblies and Analysis Systems

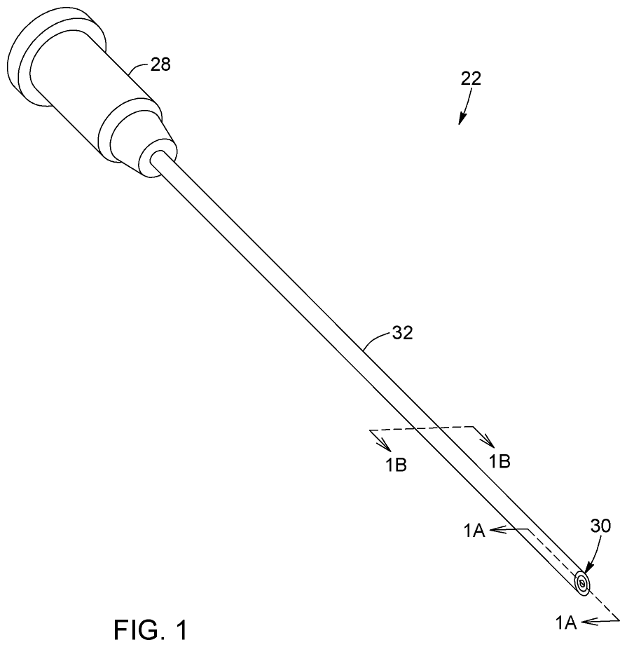

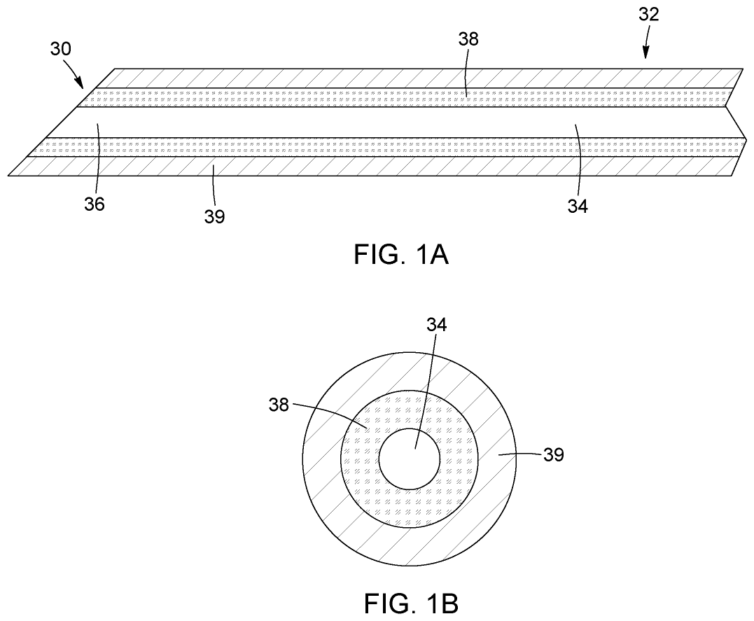

Referring to FIGS. 1, 1A and 1B, a needle assembly 22 according to one embodiment is shown. The needle assembly 22 of this embodiment includes a needle hub 28 and a needle tip 30. A shaft portion 32 extends and is disposed longitudinally between the needle hub 28 and the needle tip 30. The shaft portion 32 includes a cavity 34 extending from the needle hub 28 to the needle tip 30. The cavity 34 may be understood as an empty channel extending the entire length of the shaft portion and opened to air at both extremities. The cavity 34 includes a sample receiving region 36 opened at the needle tip 30. The needle assembly 22 according to the present embodiment may therefore be used to collect, within the sample-receiving region 36, a biological sample from a biological medium and perform an optical interrogation of this sample directly in the needle assembly 22.

In this variant, the shaft portion 32 further includes a cladding structure 38 surrounding the cavity 34. In some variants the cladding structure may be made of or include at least one optical cladding layer made of SiO₂ or other optical material suitable for light propagation and guiding. As illustrated in examples described further below, in some configurations the shaft portion of the needle assembly 22 may be configured for light guidance therein along a longitudinal optical axis to perform an optical interrogation of the sample in the sample-receiving region 36. Still in the illustrated embodiment of FIGS. 1, 1A and 1B, the shaft portion 32 may further include a sheath 39 surrounding the cladding structure 38. The sheath 39 preferably lends rigidity and solidity to the needle assembly 22 and may for example be made of metal or another robust and biocompatible material. Preferably, the sheath 39 is made of a biologically compatible material if relevant to the intended use. The sheath 39 may have a beveled extremity defining the needle tip 30. In some embodiments, the sheath 39, the cavity 34 and the cladding structure 38 are jointly beveled at the needle tip 30, such that their respective endfaces extend in a same plane. In another variant the cavity 34 and cladding structure 38 may be slightly recessed within the sheath 39 at the needle tip 30, inasmuch as such a configuration does not impede the drawing of the sample in the sample-receiving region 36.

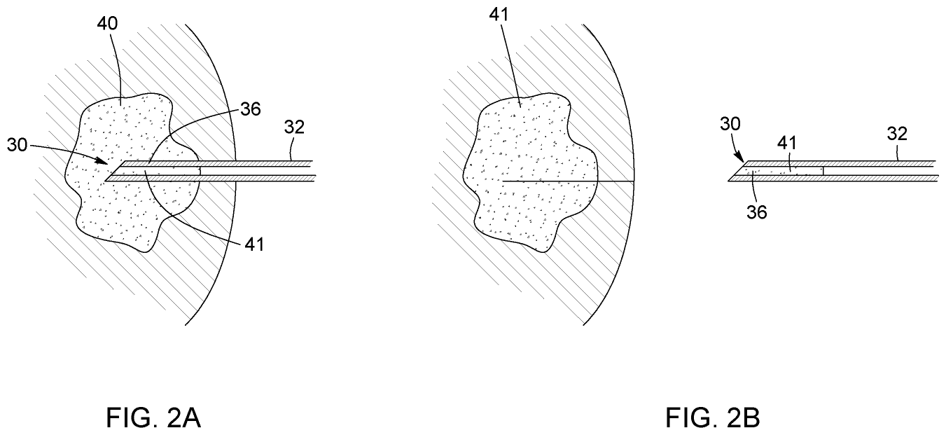

FIGS. 2A and 2B illustrate the drawing of a biological sample 41 from a biological medium 40 using a needle assembly such as shown in FIG. 1. This may for example be achieved by inserting the needle tip 30 into the biological medium 40 from which the sample is to be collected, and drawing the sample inside the sample receiving region 36 (FIG. 2A). In some variants, the simple insertion of the needle tip 30 in the biological medium 40 may suffice to collect a suitable quantity of biological material inside the needle assembly 22, this quantity defining the biological sample 41. For example, liquid samples may enter the needle tip through capillary action. Tissues may require repetitive small “stabbing” passes, typically leaving the needle tip within the medium 40 with the needle to push tissue up into the needle mechanically. In other variants, it may be desired to apply a suction force, which may for example be provided by a syringe-type device to which the needle assembly 22 is connected. Some tissues may require both a repetitive motion and suction. It will be noted that in the illustrated embodiment the sample receiving region 36 is simply embodied by the front portion of the cavity 34, that is, the portion of the cavity 34 closer to the needle tip 30. In alternative embodiments (not shown) the sample receiving region 36 may have a shape that differs from the rest of the cavity 34.

Optionally, once the biological sample 41 has been drawn into the sample receiving region 36 of the cavity 34, the needle assembly 22 may be removed from the biological medium 40, as shown in FIG. 2B. In other variants, the needle assembly 22 may remain in the biological medium 40 (from a cell culture, or a patient's body sampling site) during the optical interrogation process.

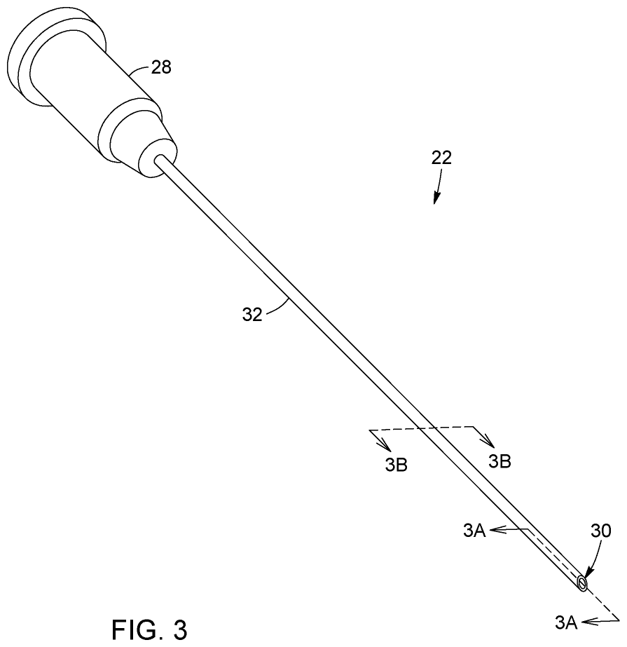

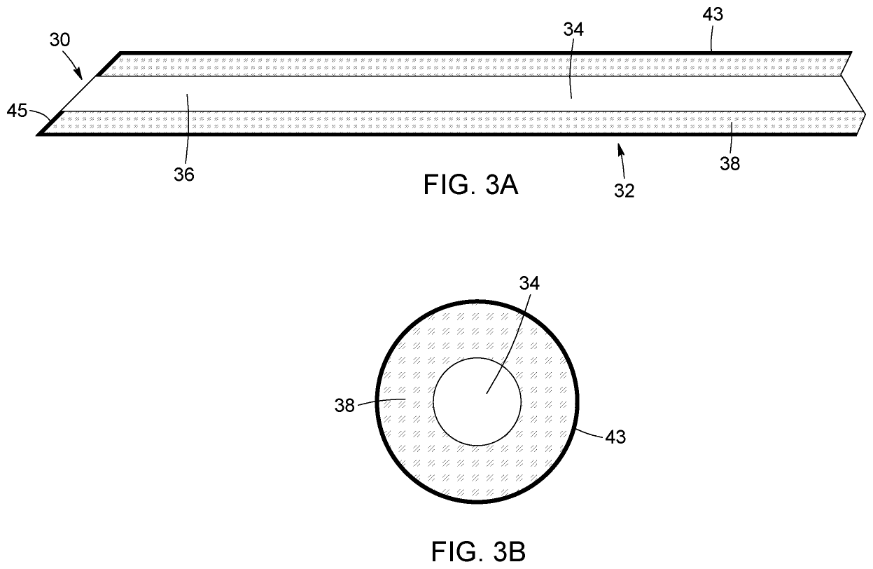

Referring to FIGS. 3, 3A and 3B, there is shown another variant of a needle assembly 22. Again, the needle assembly includes a needle hub 28, a needle tip 30 and a shaft portion 32 therebetween. The shaft portion includes a cavity 34 surrounded by a cladding structure 38, both as explained above. The cavity 36 includes a sample-receiving portion 36. This variant differs from the one illustrated in FIG. 1 by the absence of a sheath 39. In this variant, the cladding structure 38 may have a beveled extremity 40 defining the needle tip 30. The cladding structure 38 of this embodiment is preferably provided with one or more coating layers 43 to improve its rigidity, biocompatibility, optical properties, etc. In one example the coating layer or layers may include a structural coating made of a material sufficiently resistant to prevent breaking of the needle assembly 22 during the sample collection process. The one or more coating layers 43 may for example include a polyimide layer. As well known in the art, polyimide may be coated on optical fibers for medical use, as it is biocompatible and suitable for sterilization, and can provide strength and temperature resistance to the fiber. Other coating materials such as acrylate, low-index polymers, silicon and metal may also be considered. These coating materials may also be further jacketed.

In some embodiments, such as for example shown in FIG. 3A, a reflective coating 45 may be deposited on an extremity of the cladding structure 38 at the needle tip 30. The reflective coating 45 may for example be useful to reflect light back towards the needle hub 28 for extraction and detection after its interaction with the biological sample.

Various configurations may be envisioned for the cladding structure 38 without departing from the scope of the present invention.

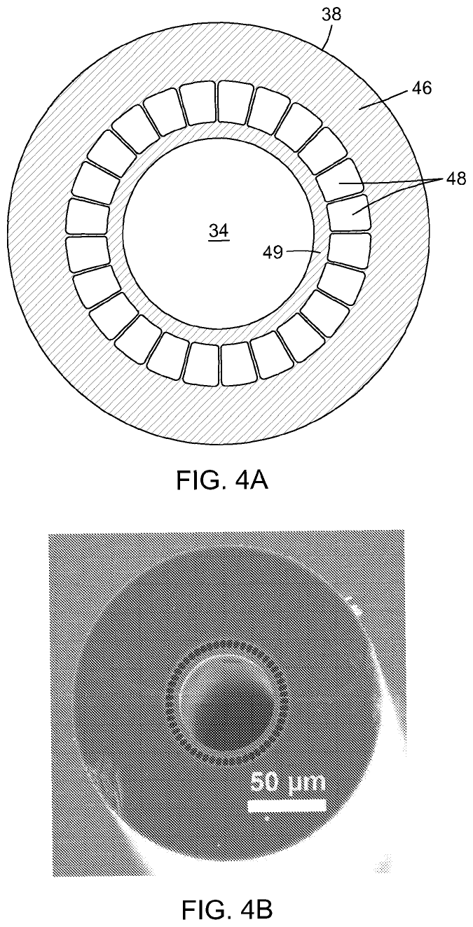

Referring to FIGS. 4A and 4B, in one embodiment the cladding structure 38 may include a silica layer 46 and an air hole layer 48 within this silica layer 46, defining an air-clad configuration. The low refractive index of the air filling the holes of the air hole layer 48 and the cavity 34 allows the light to propagate in an interstitial ring 49 of the silica layer 46 extending between them.

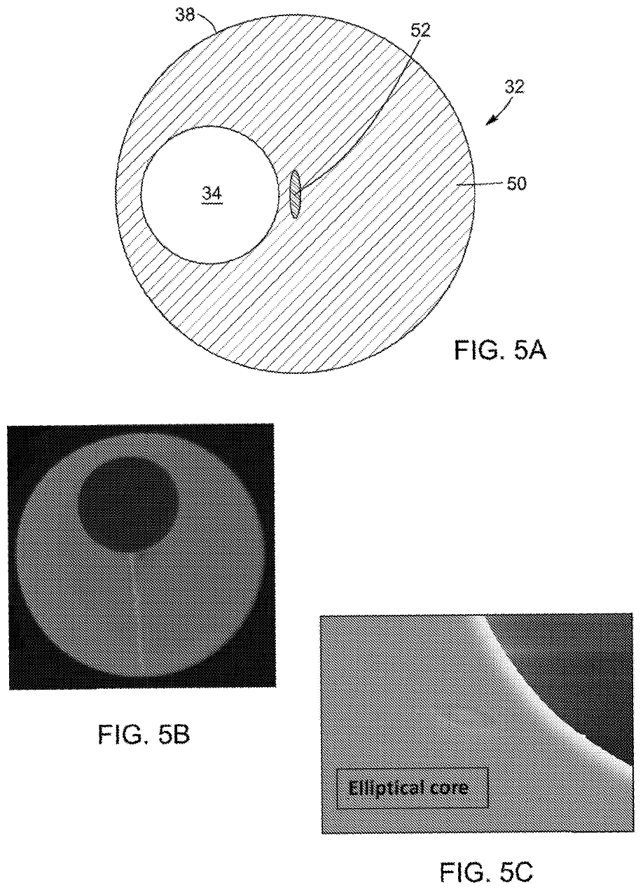

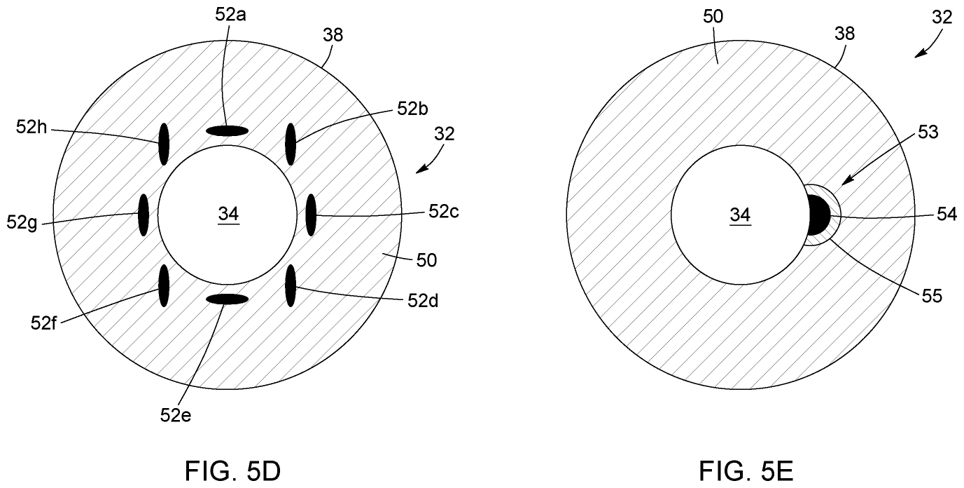

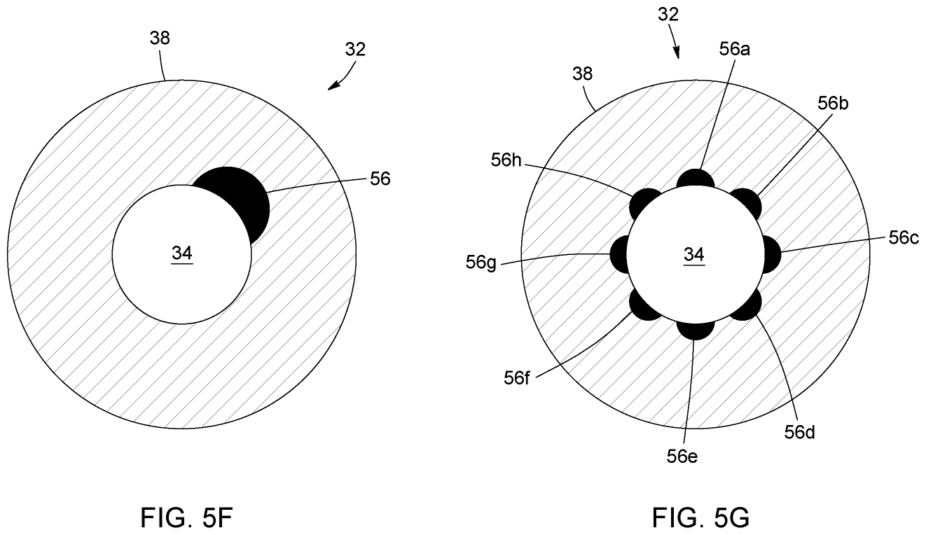

Referring to FIGS. 5A to 5C, there is shown another example of a cladding structure 38, including a silica layer 50 surrounding the cavity 34. In this variant, the cavity 34 is positioned eccentrically with respect to the central axis of the shaft portion 32. An elliptical optical fiber core 52 extends concentrically within the silica layer 50, along the central axis of the shaft portion and therefore parallel to the cavity 34. The elliptical optical fiber core 52 guides light from the needle hub to the needle tip and evanescent wave coupling can for example occur between the travelling light and the sample present in the sample-receiving region of the cavity 34. An example of such a fiber is for example shown in U.S. Pat. No. 7,405,673 (CARON et al), the entire contents of which is incorporated herein by reference. In other variants, the optical fiber core 52 may be circular or having another shape than elliptical. In another variant, as for example illustrated in FIG. 5D, several elliptical cores 52a, 52b, 52c, , 52h may be distributed around the cavity 34 and therefore provide an array of light paths surrounding the cavity 34 and the sample within. Such a variant may be used with a cavity 34 concentrically disposed within the shaft portion 32. Of course, it will be understood by one skilled in the art that the number, shape and distribution of the elliptical cores may vary and that the configuration shown in FIG. 5D is provided by way of example only. Referring to FIG. 5E, in yet another variant the cladding structure may include an integrated optical fiber 53 extending along the cavity 34. The integrated optical fiber 53 has an optical fiber core 54 and an optional optical fiber cladding 55 configured for guiding light within the optical fiber core 54. The optical fiber core 54 may for example be made of pure SiO₂ or SiO₂ doped with a material having a higher refractive index such as GeO₂, P₂O₅, TiO₂, etc. while the optical fiber cladding 55 can be made of SiO₂ doped with a lower-index material such as fluorine or B₂O₃. The optical fiber cladding 55 may be omitted if the refractive index of the optical fiber core 54 is higher than that of the cladding structure 50. The integrated optical fiber 53 is polished along its length on one side and positioned such that the optical fiber core 54 is exposed to the cavity 34 and therefore to the sample within.

Referring to FIGS. 5F and 5G, in other implementations the cladding structure may include one or more partial optical fiber cores 56 or 56a, 56b, . . . 56h extending along the cavity 34 and exposed to the cavity 34 and to the sample within. The partial optical fiber core or cores 56 may for example be made of SiO₂ doped with higher-index materials such as GeO₂, P₂O₅, TiO₂, etc. Of course, the number of partial cores 56 and their configuration may vary.

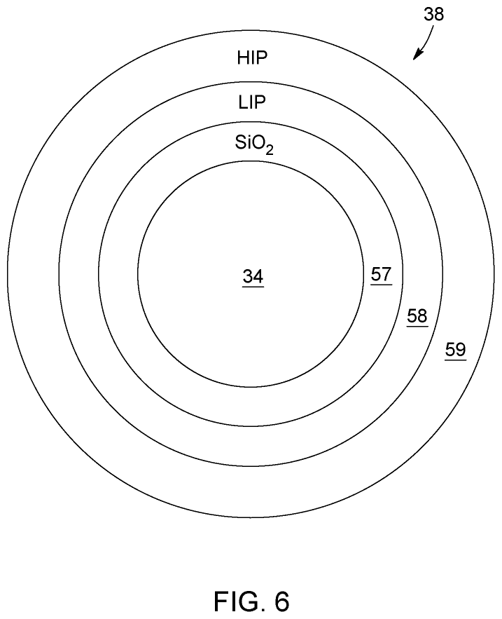

Referring to FIG. 6, in another example, the cladding structure 38 is multi-layered, that is, it is made up of a plurality of concentric cladding layers. In the illustrated example these layers include, concentrically and outwardly from the cavity 34: a ring core layer 57, for example made of SiO₂, a low refractive index cladding layer 58 and a high refractive index cladding layer 59. As will be readily understood by one skilled in the art, such a configuration may guide light within the ring core layer 57. In another variant with a similar configuration, the cladding structure surrounding the silica ring core may include a cladding layer made of SiO₂ doped with F surrounded by a polyimide or other coating layer.

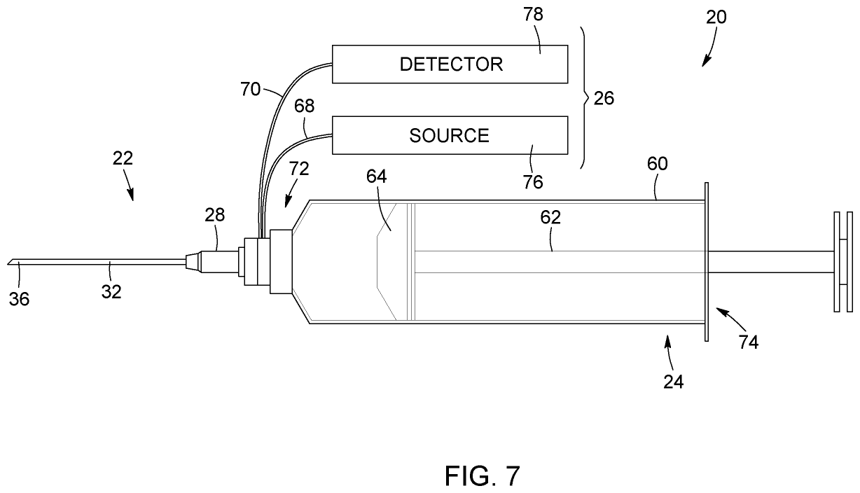

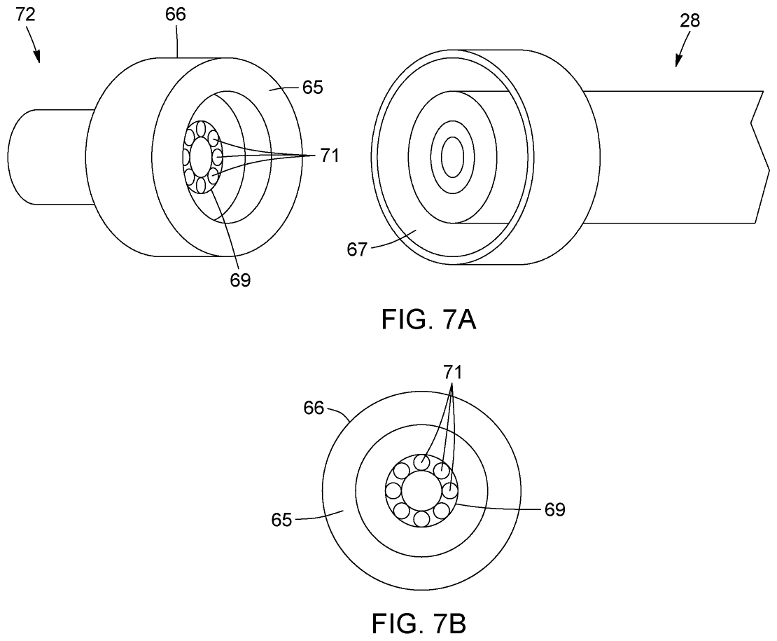

Referring to FIG. 7, in accordance with one aspect, there is provided a sample analysis system 20 for collection and optical interrogation of a biological sample.

The sample analysis system 20 first includes a needle assembly 22 according to any one of the variants described above or equivalents thereof.

In some variants, the sample analysis system 20 may further include a syringe assembly 24 connectable to the needle hub 28 of the needle assembly 22. The syringe assembly 24 can be used to provide a suction force to draw the biological sample into the sample receiving region 36 of the needle assembly 22. The syringe assembly 24 may be of standard construction, and preferably includes a barrel 60. The barrel 60 typically has a cylindrical shape and has a proximal end 72 connectable to the needle hub 28 and an open distal end 74. The syringe assembly 24 further includes a plunger 62 inserted in the barrel 60 from the distal end 74 and slideable longitudinally within the barrel 60. The end of the plunger 62 extending within the barrel 60 is provided with a plunger seal 64, creating a seal with the inner wall of the barrel 60. As well known in the art, when the needle assembly 22 is connected to the proximal end 72 of the syringe assembly 24 the movement of the plunger 62 within the barrel 60 provides the suction force which can draw the biological sample into the sample receiving region 36 of the needle assembly 22.

The syringe assembly may be connected to the needle hub 28 in a variety of manners. By way of example, a “Luer-lock” (trademark) type connection may be provided in which the proximal end 72 of the barrel 60 is provided with a male connection fitting and the needle hub with an associated female fitting. Typically, a tabbed hub on the female fitting screws into threads in a sleeve on the male fitting to provide a secure engagement. In another variant, a “Luer-slip” (trademark) or “slip tip” engagement can be provided where the male and female fittings are pressed together without involving threads.

It will be readily understood that some embodiments of the sample analysis system may exclude a syringe assembly, for example in variants where the sample is to be drawn in the sample-receiving region through capillary action or mechanically pushed therein.

The sample analysis system 20 further includes an optical assembly 26. The optical assembly 26 first includes a light source 76 generating an interrogation light beam. The interrogation light beam may have any optical characteristics suitable in view of the type of optical testing to be made on the sample. For example, for visible or near-infrared brightfield or fluorescence microscopy a white light source may be used, or a laser or LED emitting for instance at 488 nm, 532 nm, 568 nm, 633/647 nm or 676 nm. Optical sources emitting light of wavelengths centered at 800 nm, 1050 nm or 1310 nm are typically used for near-infrared optical coherence tomography. Lasers or LED sources emitting at a suitable wavelength may also be used for Raman spectroscopy (e.g. at 785 nm, 830 nm, 980 nm, 1064 nm) or for autofluorescence measurements (e.g. at 308 nm, 337 nm, 360 nm, 425 nm). Diffuse reflectance spectroscopy may also be performed with a broadband or white light source emitting light with a wavelength spectrum lying somewhere in the 400-1000 nm range (e.g. a tungsten or xenon lamp, or a combination of broadband LEDs to cover this wavelength range either partially or fully). Refractive index measurements, for example, by evanescent wave sensing, may also be performed at one or more visible or near infrared wavelengths generated by a laser or LED. Of course, it will be readily understood that the types of light sources and corresponding spectral information listed above is given by way of example only and is in no way considered limitative to the scope of the invention.

In some implementations, the light source 76 may be connectable to the needle hub 28 so as to inject the interrogation light beam into the needle assembly 22 for propagation towards the biological sample when drawn into the sample receiving region 36. The interrogation light beam may be injected for propagation in different components of the shaft portion 32 depending on the construction of the shaft portion 32 and of the interrogation scheme being applied, as will be further explained below. One or more input optical fiber links 68 may be provided to guide the interrogation light beam from the light source to the needle assembly 22.

The optical assembly 26 may further include an optical detector 78. In the implementation shown in FIG. 7, the detector 78 is connectable to the needle hub 28 to collect the light travelling in a backward direction in the needle assembly 22.

The detector 78 may be embodied by various devices, depending on the nature of the optical analysis to perform. For example, a spectrometer may be used in the context of Raman spectroscopy, diffuse reflectance spectroscopy, multi-wavelength refractive index sensing, spectral-domain optical coherence tomography, etc. Photomultiplier tubes may be used for microscopy and single emission wavelength fluorescence measurements, whereas CCD or CMOS cameras may be useful for various microscopy and imaging applications. One or more output optical fiber links 70 may be provided to guide the light resulting from an interaction of said interrogation light beam with the biological sample from the needle assembly 22 to the detector 78.

With reference to FIGS. 7A and 7B, in some implementations the optical assembly 26 may include an optical coupler 66 connectable to the needle hub 28. Preferably, the optical coupler 66 provides an optical connection between the shaft portion of the needle assembly and the optical assembly 26. The optical coupler 66 may for example be engageable in a locking engagement through a first connector 65 and a second connector 67 embodying a male-female “Luer-lock” or “Luer-slip” connection such as mentioned above. The optical coupler 66 may include light guides or otherwise provide for the propagation of light towards the needle assembly. In some implementations, the first connector 65 may be affixed to the needle hub and the second connector 67 is engageable with the first connector and houses extremities of the input and output optical fiber links. In the illustrated embodiment, for example suitable for connection to a needle assembly having a cladding structure such as shown in FIG. 6, the optical coupler 66 includes a light ring 69 being sized, shaped and positioned to provide optical coupling with the ring core layer of the cladding structure. The light ring 69 may be for example embodied by circularly disposed endfaces 71 of optical fibers, which may for example be composed of the input optical fiber links (for coupling light from the light source into the needle assembly), or the output optical fiber links (for coupling light from the needle assembly to the detector), or a combination of both.

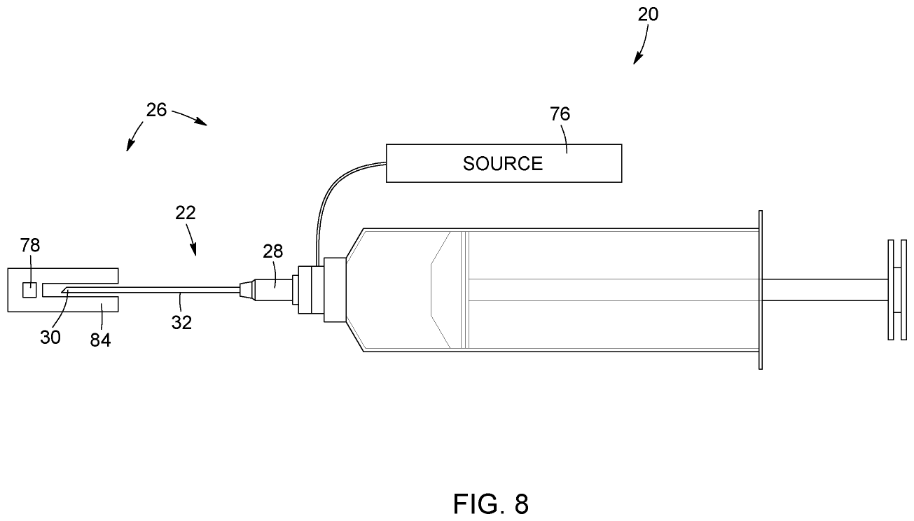

Referring to FIG. 8, there is shown another implementation of a sample analysis system 20 including an optical assembly 26. The optical assembly 26 again includes a light source 76 which may be configured according to any suitable embodiment such as for example described above, and an optical detector 78. In this variant, the detector 78 is provided within an optical reader 84 which is a component separate from the needle assembly 22. In this configuration, the needle tip 30 may be inserted into the optical reader 84, which may for example take the shape of a cap. Light is injected into the shaft portion 32 of the needle assembly 22 at the needle hub 28, and propagates towards the needle tip 30, interacting with the sample along the way. The detector 78 is affixed within the cap and positioned to detect light which exits from the needle tip 30, so that the impact of the interaction of the propagating light with the sample can be measured.

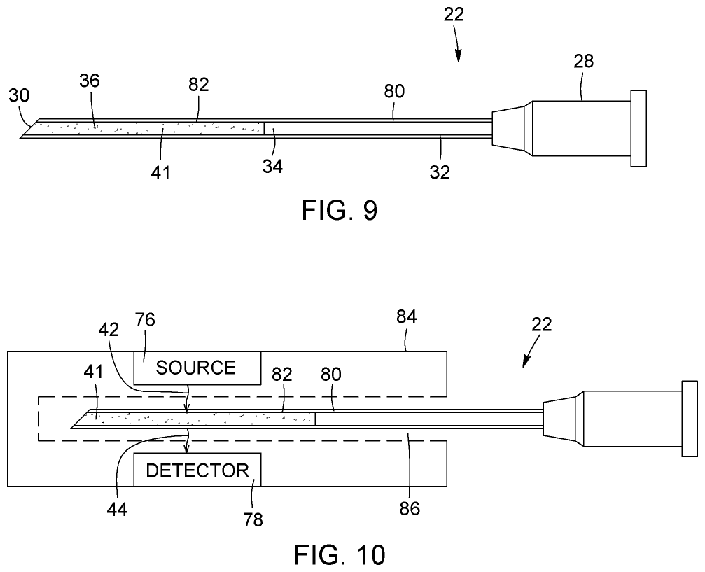

Referring now to FIG. 9, there is shown a needle assembly 22 for collection and optical interrogation of a biological sample according to another embodiment.

In this embodiment, the needle assembly 22 includes a needle hub 28, a needle tip 30 and a shaft portion 32 disposed longitudinally between the needle hub 28 and the needle tip 30. The shaft portion 32 includes a capillary 80 having a cavity 34 extending longitudinally from the needle hub 28 to the needle tip 30. The cavity 34 has a sample receiving region 36 opened at the needle tip 30, similarly to described above. The shaft portion 32 further includes at least one optical window 82 transversally aligned or in line of sight alignment with the sample-receiving region 36 and allowing optical interrogation of the sample within the sample receiving region 36 therethrough. In the illustrated variant of FIG. 9, the shaft portion 32 of the needle assembly 22 entirely consists of the capillary 80, which is made of a transparent material. The optical window 82 thus corresponds to the portion of the capillary 80 that defines the sample receiving region 36. In other embodiments (not shown), the optical window or windows may extend over a portion only of the shaft portion, for example as an opening or insert through the capillary. In this context, it will be understood that the positioning of the optical window or windows with respect to the sample-receiving region may be offset from a direct alignment inasmuch as light may travel inwards through the optical window towards the biological sample on the one hand, and outwards from the sample to exit the needle assembly substantially unobstructed or unattenuated on the other hand. In some embodiments the light may travel in both directions through a same optical window. In other embodiments light may cross different optical windows in the input and output directions.

The needle assembly 22 according to the present embodiment may be used to collect a biological sample 41 from a biological medium (from a cell culture, or a target sampling site) and perform an optical interrogation of this sample 41 directly in the needle assembly 22. Similarly to the process described above with reference to FIGS. 2A and 2B, this may for example be achieved by inserting the needle tip 30 in the biological medium 40 and drawing the sample 41 inside the sample receiving region 36. In some variants, as explained above, the simple insertion of the needle tip 30 in the biological medium 40 may suffice to collect a suitable quantity of biological material inside the needle assembly 22, either by capillary action or through repetitive “stabbing”. In other variants, it may be desired to apply a suction force, which may for example be provided by a syringe-type device. It will be noted that in the illustrated embodiment the sample receiving region 36 is simply embodied by the front portion of the hollow fiber core 34, that is, the portion of the cavity 34 closer to the needle tip 30. In alternative embodiments the sample receiving region 36 may have a different shape than the rest of the cavity 34.

Once the biological sample 41 has been drawn into the sample receiving region 36 of the cavity 34, the needle assembly 22 is removed from the biological medium 40. Optical interrogation of the sample 41 is then performed by projecting one or more interrogation light beams towards the sample 41 through the optical window 82 or windows. Light resulting from the interaction of the interrogation light beam with the sample can be transmitted light exiting the needle assembly through the needle tip, or light reflected or otherwise travelling backwards with respect to the direction of the optical interrogation. It will be readily understood that the reference to a transversal optical interrogation includes impinging and transmitting the interrogation light beam through the optical window at various possible incidence angles differing from a purely longitudinal light propagation scheme and is not limited to light injection at a right angle with respect to the longitudinal axis of the shaft portion. Advantageously, in some embodiments this variant may provide the ability to image or enable spatially distributed sampling.

Referring to FIG. 10, there is shown a portion of a sample analysis system including a needle assembly 22 such as shown in FIG. 9. In this implementation, the analysis system 20 includes an optical reader 84, and optical interrogation of the sample involves inserting the needle tip 30 and the part of the shaft portion which includes the sample-receiving region into the optical reader 84. The optical reader 84 may for example have a portion forming a reading chamber 86 sized to receive the needle tip 30. The optical reader 84 preferably includes at least one light source 76 and at least one optical detector 78. The light source or sources are configured to generate one or more interrogation light beams 42 having suitable optical properties for the type of analysis to be performed on the sample 41. As known to those skilled in the art, the interaction of the interrogation light beam(s) 42 with a sample leads to the generation of either return or transmitted light having optical properties representative of characteristics of the sample 41 and can be analyzed through various techniques to yield information about the sample. As will be readily understood by one skilled in the art, the optical reader 84 may also include a moving source/detector assembly or several sources and detectors for distributed analysis along the length of the needle or for multi-spectral analysis or any other analysis method.

In the illustrated embodiment of FIG. 10, the light sources and detectors (one or many of each) are aligned or otherwise positioned such that an interrogation light beam 42 or beams can be generated by the light sources and traverse the sample, light 44 resulting from the interaction of the interrogation light beam with the sample being detected by the detector on the opposite side of the sample. While FIG. 10 shows a direct line-of-sight alignment between the light source and detector, it will be readily understood by one skilled in the art that in other implementations (not shown) either the interrogation light beam 42 or the transmitted light beam may be redirected, by mirrors, lenses and the like. Furthermore, it will be readily understood that other optical components directing, shaping, modulating or otherwise affecting either the interrogation light beam 42, the transmitted light beam or both may be provided within the reader 84 without departing from the scope of the invention. In other variants (not shown), the light source and the detector may be positioned on a same side of the sample such that light reflected, scattered, re-emitted or otherwise returning in the direction from which the interrogation light beam 42 impinged the sample is collected and analyzed. The optical reader 84 may take various shapes, and in some embodiments may be formed as a cap which can be placed over the needle tip similarly to the variant shown in FIG. 8. It will further be understood that in some embodiments the optical reader 84 as described above may be used in conjunction with a needle assembly defining a light guiding structure such as described with respect to FIG. 1 or the like, provided that the cladding structure of the needle assembly is sufficiently transparent or includes an optical window allowing optical interrogation therethrough.

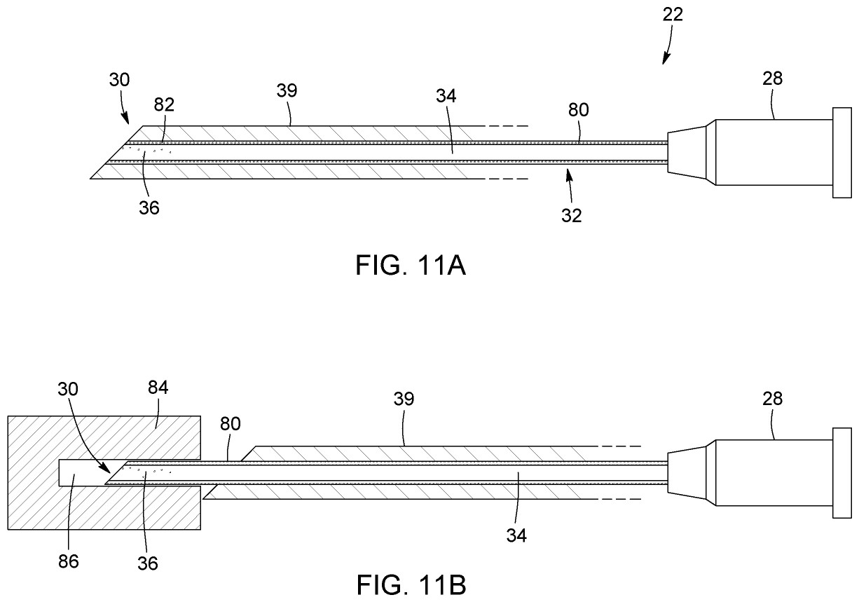

Referring to FIGS. 11A and 11B, there is shown another variant of a needle assembly 22 that can be used for transversal interrogation of the sample within the sample-receiving region 36, either in a light transmission mode or in a light reflectance mode. The needle assembly 22 of this embodiment includes a needle hub 28, a needle tip 30 and a shaft portion 32 between the needle hub 28 and the needle tip 30. The shaft portion 32 includes a capillary 80 having a longitudinal cavity 34 extending from the needle hub 28 to the needle tip 30. The cavity 34 has a sample receiving region 36 opened at the needle tip 30, similarly to what has been described above. The shaft portion 32 includes an optical window 82 transversally aligned with the sample-receiving region 36 so as to allow optical interrogation of the sample within the sample receiving region 36 transversally to the longitudinal axis of the shaft portion 32. Preferably, the capillary 80 is entirely made of a transparent material, inherently embodying an optical window 82. The shaft portion 32 further includes a sheath 39 surrounding the capillary 80. The sheath 39 preferably lends rigidity and solidity to the needle assembly 22 and may for example be made of metal, polyimide or an equivalent material. Preferably, the sheath 39 is made of a biologically compatible material if relevant to the intended use. In the illustrated embodiment the sheath 39 is retractable and has a sampling position (see FIG. 11A) where its front extremity is flush with the front extremity of the capillary 80, thereby enabling drawing of the sample inside the sample receiving region 36. The sheath 39 also has a retracted position (see FIG. 11B) wherein it is retracted with respect to the needle tip 30 in order to allow the needle tip 30 and sample-receiving region 36 to be exposed. The sheath may be affixed in the sample-receiving position during the sampling process, and the reader 84 may be configured so that insertion of the needle tip therein pushes the sheath in the retracted position, such that the sample-receiving region is exposed for optical interrogation of the sample as explained above. In such an implementation the sheath is preferably shorter than the shaft portion of the needle assembly, and additional protecting and/or blocking implements may be provided around the shaft portion at the extremity of the needle hub when the sheath is in the sample-receiving position. In some implementations, the optical reader may include a recess or other means for guiding the needle tip in the reading chamber 86 and avoid breakage of the capillary in the process of inserting the needle tip in the reader.

In a different variant (not shown), the sheath 39 may be designed such that optical interrogation is allowed through this sheath 39, either in transmission or in reflectance. For example, the sheath 39 may be made of an optically transparent material. Alternatively, the sheath 39 may have one or more openings or transparent inclusions defining the optical window 82 and allowing optical access to the sample receiving region 36.

### Examples of Methods for the Diagnosis, Prognosis or Treatment of a Disease or a Condition

In accordance with some implementations, needle assemblies and analysis systems such as described above or equivalents thereof may be used in different contexts.

In some embodiments, there is provided a use of a needle assembly according to variants as defined above, and the like, for an in situ optical interrogation of a biological sample of a subject. Such a use involves the sample being within the sample-receiving portion of the needle assembly and optical interrogation longitudinally along the needle assembly, while the needle assembly is still within or in contact with the sampling site.

In some embodiments, there is provided a use of a needle assembly according to any of the variants above, and the like, for an ex vivo optical interrogation of a biological sample of a subject. Such a use involves the sample being within the sample-receiving portion of the needle assembly during this optical interrogation.

In some implementations, there may be provided a method for analyzing a biological sample. The method may include the following steps:
obtaining a needle assembly according to an embodiment described above or the like;
inserting the needle tip of the needle assembly in a target site comprising a biological tissue or liquid;
collecting a biological sample of the target site in the sample-receiving region of the needle tip;
optically interrogating the biological sample within the sample-receiving region of the needle tip using an interrogation light beam; and
detecting and analyzing light resulting from an interaction of the biological sample with the interrogation light beam.

In some implementations the biological sample may be a tissue, such as for example normal or abnormal, e.g. malignant, tissue or cells of the breast, lymph nodes, thyroid, salivary glands, liver, pancreas, metastatic lesions. In other implementations the biological sample may be liquid such as, for example, a biological fluid, such as for example blood, lymph, tears, sweat, saliva, or urine. Alternatively, the biological sample may be liquid such as a cell suspension from a cell culture.

In some implementations, the biological sample's tissue or liquid may be collected from a body of a subject. Particularly, the subject is an animal or a human.

The optically interrogating, detecting and analyzing steps of the method may be carried out in a variety of fashions, depending on the desired information, structure of the needle assembly and capabilities of the components of the analysis system.

In some examples, for example using a needle assembly such as or equivalent to those described in FIGS. 1, 3, 4A, 4B, 5A through 5G and 6, the optical interrogation of the sample may involve propagating the interrogation light beam longitudinally in the needle assembly. For example, the interrogation light beam may be injected into the needle assembly at the needle hub and propagates towards the needle tip. The interrogation light beam may be guided or otherwise propagate along an interstitial ring, a ring core, one or more elliptical optical fiber core, one or more integrated optical fiber, one or more partial optical fiber cores, the cavity or the like. The light resulting from an interaction of the biological sample with the interrogation light may be collected at the needle tip or at the needle hub. In some implementations the optical interrogation is performed subsequently to the removal of the needle tip from the body part. In other variants the optical interrogation may be performed in vivo, after collection of the sample from the subject but while the needle tip is still within the body part of the subject.

In alternative examples, for using a needle assembly such as or equivalent to those described in FIGS. 9, 11A and 11B, optical interrogation of the sample may involve propagating the interrogation light beam towards the sample transversally to the needle assembly. In some variants, the interrogation light beam may enter the needle assembly through one side of the shaft portion and the resulting light exits from the opposite side. In other variants the resulting light may exit the needle assembly on the same side from which the interrogation light beam entered or at any angle. Both these sets of variants are understood to fall within the scope of “transversal” optical interrogation. Light may enter and exit the needle assembly at different angles with respect to the longitudinal axis of the shaft portion.

In various embodiments, the interrogation light beam may be absorbed, scattered or transmitted by the biological sample. In some embodiments, the interrogation light beam may interact with the sample through evanescent wave coupling from a waveguiding core parallel to the cavity (such as for example the elliptical optical fiber cores of FIGS. 5A and 5D).

In some implementations, the analysis of the light resulting from the interaction of the interrogation light beam with the sample may provide one or more information of different types, such as for example:
The presence of a sample within the sample receiving region and/or the quantity of biological material present within the sample receiving region;
Adequacy/cellularity information. This may for example take the form of the amount of cells, density or ratio with respect to the entire volume collected which may contain unwanted fluids like blood, lymph or other biofluids. In some implementations, adequacy/cellularity information may include an indication of whether or not enough of the sample has been collected to make a diagnosis. If not, the pathologist may receive the sample and classify it as ‘inadequate’ for diagnosis. In other implementations, adequacy/cellularity information may include a measure of the refractive index of the sample, e.g. 1.33 for water, 1.37 for blood and 1.4 for tissue. This information may also involve the density of the sample by scattering (OCT or reflectance spectroscopy), the use of microscopy to count cells or measure cellular density, etc. In other variants, the present method may be used to confirm that the collected tissue is indeed the target tissue. Such a variant may require more specific measurements, such as Raman spectra or microscopic features enabling the differentiation of a target lesion with respect to the surrounding tissue.
The nature of the sample or properties thereof such as malignant or benign, etc. This type of information may for example be obtained through spectroscopic analysis such as Raman spectroscopy, fluorescence, etc.

In some embodiments, there is also provided a use of the needle assembly according to any of the variants above, and the like, for an ex vivo optical analysis of a biological sample of a subject to diagnose a disease or condition in the subject, the sample being within the sample-receiving portion of the needle assembly during the optical analysis.

In some implementations, there may be provided a method to aid in diagnosis, prognosis or guide treatment of a disease or a condition in a subject, comprising the steps of:
obtaining a needle assembly according to an embodiment described above or the like;
inserting the needle tip of the needle assembly in a body of a subject such that the needle tip reaches a target site; and
collecting a biological sample from the target site in the sample-receiving region of the needle assembly;
optically interrogating the biological sample within the sample-receiving region of the needle tip using an interrogation light beam;
detecting light resulting from an interaction of the biological sample with the interrogation light beam;
analyzing the resulting light to determine at least one characteristic specific of said disease or condition; and
transmitting at least one characteristic specific of the disease or condition to a physician.

The sampling site may be, for example, a biological sample that is outside of its natural environment, such as in vitro or, alternatively, in a cell culture environment.

Alternatively, the sampling site is a body of a subject as defined above. The sample's tissue or cells may therefore be collected from a body part such as, for example, a body member (e.g. arm, leg, trunk, head), an organ such as breast, lymph nodes, thyroid, salivary glands, liver, pancreas, a specific tissue (e.g. metastatic lesions), or a biological fluid such as blood, urine, lymph, tears, saliva or sweat.

The disease or condition of the subject may for example be cancer, diabetes, malaria, or other conditions in which the cells, tissues or biofluids have optical properties differing from those usually observed with healthy cases.

Example characteristics measurable with optical techniques and specific of the disease or condition may for example be increased nucleus/cell size and/or decreased extracellular tissue organization in the case of cancer, the presence of proteins or blood cells in urine in the case of diabetes, spectral or structural changes in blood cells due to malaria infection, etc.

The step of collecting a biological sample comprises drawing the biological tissue or cells within the sample-receiving portion, for example using a syringe assembly connected to the needle hub or the mechanical stabbing method described above, with or without a syringe for suction.

The method may include a step of withdrawing the needle tip from the body of the patient between the steps of collecting the biological sample and optically interrogating the biological sample.

As mentioned above, in other variants the optical interrogation may be performed in situ, after collection of the sample from the sampling site or the body part of the subject but while the needle tip is still within the tissue, cell culture or body of the subject. A one skilled in the art will readily understand, such variants are of interest when using a configuration of the needle assembly allowing the interrogation light beam to propagate longitudinally in the needle assembly.

The analyzing step of the present method for the diagnosis, prognosis or treatment of a disease or a condition in a subject may involve using an optical analysis technique such as visible or near-infrared brightfield or fluorescence microscopy, visible or near-infrared optical coherence tomography, Raman spectroscopy, autofluorescence measurements, diffuse reflectance spectroscopy and refractive index measurements, and the like.

It will be readily understood that while the present methods advantageously provide for the optical interrogation of the sample while it is within the sample receiving region of the needle assembly, in some implementations the sample may subsequently be extracted from the needle assembly and processed or further analyzed according to techniques known in the art in order to obtain further information from this sample.

Of course, numerous modifications could be made to the embodiments described above without departing from the scope of the invention.

1-78. (canceled)

79. A needle assembly for collection and optical interrogation of a biological sample, comprising:
a needle hub;
a needle tip; and
a shaft portion disposed longitudinally between the needle hub and the needle tip, the shaft portion comprising a capillary having a cavity extending longitudinally from the needle hub to the needle tip, the cavity comprising a sample-receiving region opened at the needle tip for collection of the biological sample through the needle tip, the capillary further comprising at least one optical window in line of sight alignment with the sample-receiving region and allowing optical interrogation of the sample within the sample-receiving region therethrough.

80. The needle assembly according to claim 79, wherein the capillary is made of a transparent material, a portion thereof defining each of the at least one optical window.

81. The needle assembly according to claim 80, wherein the transparent material is silica or a plastic.

82. The needle assembly according to claim 79, wherein the at least one optical window comprises an input window and an output window.

83. The needle assembly according to claim 82, wherein the input and output windows are provided on opposite sides of the capillary in optical alignment.

84. The needle assembly according to claim 79, further comprising a sheath made of a biologically compatible material surrounding the shaft portion.

85. The needle assembly according to claim 84, wherein the biocompatible material comprises a metal or a polyimide.

86. The needle assembly according to claim 84, wherein the sheath is movable over the capillary between a sampling position enabling drawing of the sample inside the sample-receiving region and a retracted position exposing the at least one optical window to optical interrogation therethrough.

87. The needle assembly according to claim 84, wherein the sheath is made of a transparent material.

88. The needle assembly according to claim 84, wherein the sheath comprises at least an opening or transparent inclusion optically aligned with the at least one optical window of the capillary.

89. A sample analysis system for collection and optical interrogation of a biological sample, comprising:
a needle assembly according to claim 79; and
an optical reader comprising:
a reading chamber sized to receive the needle tip therein;
at least one light source generating one or more interrogation light beams and configured to project the interrogation light beams on the biological sample in the sample-receiving region through the at least one optical window when the needle tip is inserted into the reading chamber; and
at least one optical detector positioned and configured to detect light resulting from. an interaction of the biological sample with the one or more interrogation light beams.

90. The sample analysis system according to claim 89, wherein the at least one light source and the at least one optical detector are disposed on opposite sides of the reading chamber.

91. The sample analysis system according to claim 89, wherein the at least one light source and the at least one optical detector are disposed on a same side of the reading chamber.

92. The sample analysis system according to claim 89, further comprising a syringe assembly connectable to the needle hub so as to provide a suction force to draw the biological sample into the sample-receiving region of shaft portion of the needle assembly.

93. A method for analyzing a biological sample, comprising the steps of:
obtaining a needle assembly as defined in claim 79;
inserting the needle tip of the needle assembly in a target site comprising a biological sample;
collecting the biological sample from the target site in the sample-receiving region of the shaft portion of the needle assembly;
withdrawing the needle tip from the target site;
optically interrogating the biological sample within the sample-receiving region of the shaft portion of the needle assembly using an interrogation light beam; and
detecting and analyzing light resulting from an interaction of the biological sample with the interrogation light beam.

94. A method to aid in diagnosis, prognosis or to guide treatment- of disease or a condition in a subject, comprising the steps of:
obtaining a needle assembly as defined in. claim 79;
inserting the needle tip of the needle assembly in a body of the subject such that the needle tip reaches a target site; and
collecting a biological sample from the target site in the sample-receiving region of the shaft portion of the needle assembly
withdrawing the needle tip from the target site;
optically interrogating the biological sample within the sample-receiving region of the shaft portion of the needle assembly using an interrogation light beam;
detecting light resulting from an interaction of the biological sample with. the interrogation light beam;
analyzing the light to determine therefrom at least one characteristic specific of the disease or condition; and
transmitting the at least one characteristic specific of the disease or condition to an instrument or a physician.

95. The method according to claim 93, wherein the step of optically interrogating the biological sample comprises inserting the needle tip into a reading Chamber of an optical reader.

96. The method according to claim 93, wherein the analyzing step comprises using an optical analysis technique selected from visible or near-infrared brightfield or fluorescence microscopy, visible or near-infrared optical coherence tomography. Raman spectroscopy, autofluorescence measurements, diffuse reflectance spectroscopy and refractive index measurements.

97. The method according to claim 94, wherein the step of optically interrogating the biological sample comprises inserting the needle tip into a reading chamber of an optical reader.

98. The method according to claim 94, wherein the analyzing, step comprises using an optical analysis technique selected from visible or near-infrared brightfield or fluorescence microscopy, visible or near-infrared optical coherence tomography, Raman spectroscopy, autofluorescence measurements, diffuse reflectance spectroscopy and refractive index measurements,
